(19) United States
(12) Patent Application Publication
(10) Pub. No.: US 2024/0006034 A1
(43) Pub. Date: Jan. 4, 2024

(54) SYSTEMS AND METHODS OF UTILIZING EMOTION DYADS TO DETERMINE AN INDIVIDUALS EMOTION STATE

(71) Applicant: University of North Carolina at Wilmington, Wilmington, NC (US)

(72) Inventors: Dale Cohen, Kure Beach, NC (US); Len Lecci, Wilmington, NC (US)

(73) Assignee: University of North Carolina at Wilmington, Wilmington, NC (US)

(21) Appl. No.: 18/217,152

(22) Filed: Jun. 30, 2023

Related U.S. Application Data
(60) Provisional application No. 63/357,245, filed on Jun. 30, 2022.

(51) Int. Cl. G16H 10/20

(52) U.S. Cl. CPC G16H 10/20; G16H 20/70

(57) ABSTRACT

In one aspect, a method for emotion mapping where computing device can generate an emotional dyad, wherein the emotional dyad comprises two different emotion states. The emotion dyads can be displayed with a degree of separation for receiving an input on a scale from an individual. The computing device can receive an input of a first endpoint anchor and a second input anchor on the dyad scale. Next, the computing device receives input of an emotion anchor within the degree of separation. The computing device can subsequently calculate the length of separation from the first endpoint anchor and the second endpoint anchor, the length of separation being a neutral region, and lengths outside of the neutral region being emotion regions. The computing device determines a user's assessment by averaging the neutral region, the endpoint anchors, and an emotion anchor in one or more cycles.

### RELATED APPLICATION DATA

This application claims priority under 35 U.S.C. § 119(e) to U.S. Provisional Patent Application No. 63/357,245 filed Jun. 30, 2022, the entirety of which is incorporated by reference herein.

### FIELD

The technology described herein generally relates to computer implemented systems and methods of natural language understanding and processing, more particularly to the mapping of emotion dyads using computing systems to create an assessment of an individual's emotion state.

### BACKGROUND

Measuring private events such as emotion is a difficult task. Private events consist of two independent factors. The first is the perception of an event, the second is the categorization of that bodily sensation that is converted to label the event. This data may originate in several forms, from written responses to oral, or as input to a form or field within a computing environment.

Most assessments simply measure the labeling of an event, for example, do you feel sad. These assessments are highly structured, and in part operate under rigid conditions and standards. For example, the labeling of an event is difficult to interpret without information about the bodily sensation together with the categorization parameters. For example, with regard to the emotion of “sad,” there is a bodily sensation and categorization of the emotion.

Accordingly, there is a need to enable better understanding of emotions, and how they are often perceived differently, and at different ranges. The technology described herein addresses this need and the limitations of conventional methods of emotion categorization with the assistance of computing systems and analysis of emotion dyads to advance understanding and determinations of an emotion state, and further provide improved systems and methods for assessing emotion categorization and labeling

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

At a high level, embodiments of the technology described herein are generally directed towards systems and methods for assessing emotion categorization and labeling utilizing generated bounded visual analog scales incorporating emotion dyads.

According to some embodiments, a computer implemented method for emotion mapping is disclosed. In some aspects, a computing device generates an emotional dyad, wherein the emotional dyad comprises two different emotion states. Next, the computing device displays the emotion dyads with a degree of separation for receiving input on a scale from an individual. Next, the computing device receives input of a first endpoint anchor on the dyad scale. Next, the computing device receives input of a second endpoint anchor on the dyad scale. Next, the computing device receives input of an emotion anchor within the degree of separation. Then the computing device calculates the length of separation from the first endpoint anchor and the second endpoint anchor, wherein the length of separation is a neutral region, and lengths outside of the neutral region are emotion regions, and an emotion anchor. Lastly, the computing device determines the individual's assessment by averaging the neutral region, the endpoint anchors, and the emotion anchor in one or more cycles of acquiring endpoints, emotion anchors, and determining the lengths of the neutral region and emotion region.

According to other embodiments, a system for emotion mapping and assessment thereof is provided. In some aspects, a computing device or user device is equipped with a user input device and a display device. Further, the computing device is configured with a software application held on memory. The software application can comprise: (a) a dyad engine, wherein the dyad engine generates dyad pairs; (b) an input engine, wherein the input engine accepts user input of a first anchor endpoint, a second anchor endpoint, and an emotion anchor; and (c) an assessment engine, wherein the assessment engine calculates an emotion state based on the dyad engine and the input engine.

Additional objects, advantages, and novel features of the technology will be set forth in part in the description that follows, and in part will become apparent to those skilled in the art upon examination of the following, or can be learned by practice of the invention.

### BRIEF DESCRIPTION OF THE DRAWINGS

Aspects of the present disclosure will be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. It should be recognized that these implementations and embodiments are merely illustrative of the principles of the present disclosure. In the drawings:

FIG. 1 illustrates an example of a perception of hue, and a categorization of that bodily sensation that converts to a label, in accordance with some aspects of the technology described herein;

FIG. 2 illustrates an example block diagram of an emotion mapping system, in accordance with some aspects of the technology described herein;

FIG. 3 illustrates an example of perception of bodily sensation with a dyad pair, in accordance with some aspects of the technology described herein;

FIGS. 4A-B illustrate an example of perception of bodily sensation with a dyad pair, wherein neutral regions are shown, and an emotion anchor, in accordance with some aspects of the technology described herein;

FIG. 5 illustrates an example of perception of bodily sensation with an internal consistency check shown, in accordance with some aspects of the technology described herein;

FIG. 6 illustrates an example of a block diagram of a computing device, in accordance with some aspects of the technology described herein;

FIG. 7 illustrates a flow chart of an example method for emotion mapping, in accordance with some aspects of the technology described herein;

FIG. 8A illustrates an example of an additional computing device, in accordance with some aspects of the technology described herein;

FIG. 8B illustrates a block diagram of an example computing environment and/or device architecture in which some implementations of the present technology may be employed;

FIGS. 9-13 illustrate examples of testing data acquired from systems and methods disclosed herein, in accordance with some aspects of the technology described herein; and

FIGS. 14A-14B illustrate example cut scores for generating a user assessment, in accordance with some aspects of the technology described herein.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7. All ranges disclosed herein are also to be considered to include the end points of the range, unless expressly stated otherwise. For example, a range of “between 5 and 10” or “5 to 10” or “5-10” should generally be considered to include the end points 5 and 10. Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Described herein are systems and methods for assessing emotion categorization and labeling utilizing a bounded visual analog scale (also referred to herein as a universal emotion line, emotion scale, visual scale). As will be appreciated, a visual analog scale can utilize received input from a user and further utilizes signals or information represented by continuously variable spatial position or distance indicators, for instance a spatial position and/or distance between two endpoint anchors or bounding points. The bounded visual scale or universal emotion line additionally can be implemented to measure how people categorize emotions and how that categorization related to their reported magnitude of underlying emotional affect.

According to some aspects a visual scale can be generated, by a user device or computing device) for an emotional dyad, where the emotional dyad can comprise two different emotional states. In some instances, the emotional dyad's can include: angry-compassionate; anxious-calm; cold-hot; fatigued-energized; fearful-brave; not in control-in control; powerless-powerful; sad-happy; tired-awake; uninterested-excited; unsafe-safe; and unsatisfied-satisfied. A generated emotional dyad may be presented to a user via a user device or display screen. Further, an emotional dyad and/or visual scale can include one or more indicators (e.g. two indicators) presented on the visual scale, where the indicators are displayed with an initial degree of separation. In some other aspects indicators are not initially displayed. In some instances, one or more prompts may be displayed to a user, either initially or subsequent to receiving a user input. Dyads and associated prompts may be stored in a repository from which the system can store and/or pull data from. A user can subsequently interact with the visual scale and/or emotional dyad via one or more input signals. In one instance, a user device can receive a first input anchor corresponding to one of the emotional states of the emotional dyad, where the first endpoint anchor indicates when the individual or user moves from a first emotion state to a first neutral state. A user device can receive a second input anchor corresponding to the other of the emotional states of the emotional dyad, where the second input anchor indicates when the individual or user moves from a second emotion state to a second neutral state. Additionally, a user device can further receive an input corresponding to an emotion anchor within the degree of separation. In some instances, the emotion anchor can correspond to a time-based emotion indication. The user device, based on the inputs, can calculate and/or determine the length of separation from the first endpoint anchor and the second endpoint anchor, wherein the length of separation is a neutral region, and lengths outside of the neutral region are emotion regions, as well as a location of the emotion anchor. In some instances, a device can determine a proportion corresponding to each of the lengths based on the degree of separation. Based on the determining of one or more aspects of the visual scale, a user emotion score or user/individual assessment can be determined and/or generated and subsequently compared against one or more cut scores (e.g. previously determined cut scores or cutoff scores) for a selected or given emotional dyad to determine a course of action for the user. As will be appreciated, cut scores can be stored such that they correspond to a given emotional dyad.

The present technology may be embodied as, among other things, a system, method, or computer-product. Accordingly, embodiments may take the form of a hardware embodiment, a software embodiment, or an embodiment combining software and hardware. In one embodiment, the present invention takes the form of a computer program product that includes computer useable instructions embodied on one or more computer readable media and executed by one or more processors.

Computer readable media includes both volatile and nonvolatile media, removable and non-removable media, and media readable by a database, a switch, and various other network devices. Network switches, routers, access points, and related components in some instances act as a means of communication within the scope of the technology. By way of example, computer readable media comprise computer storage media and communications media.

Computer storage media or machine readable media can include media implemented in any method or technology for storing and/or transmitting information or data. Examples of such information include computer-useable instructions, data elements, data structures, programs and program modules, and other data representations.

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

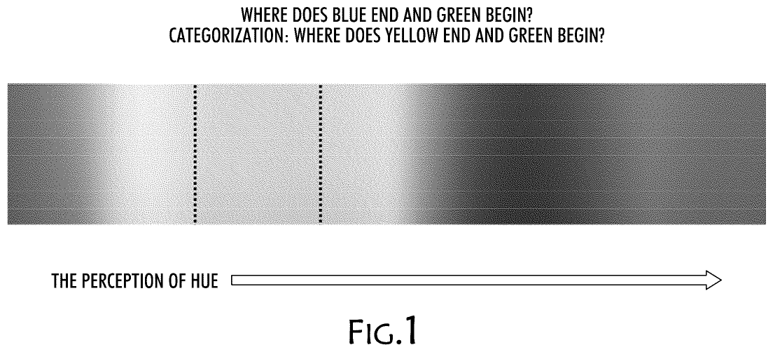

Referring now to FIG. 1, an example of a perception of hue, and a categorization of that bodily sensation that converts to a label. For example, to categorize “green” one must place a boundary somewhere, for example, between yellow and green on the depiction of FIG. 1. They must also place a boundary somewhere, for example, between blue and green. How individuals place these boundaries provides insight on how they characterize or conceptualize the color green. The same is true of emotion states, there is a bodily sensation and the categorization of each state. In one aspect a dyad is utilized with word pairings, such as happy and sad, wherein they represent separate and opposite emotions. In this aspect the computing system generates through a dyad engine a pair of separate but opposite emotions separated by a bounding box or a distance or a degree of separation in which a user may place a first anchor and a second anchor to capture their bodily sensation of the dyad pair. In this example an individual may be asked to place a tick mark on the line, or move a slider, forming an endpoint anchor indicating where the first emotion begins and moves into a neutral feeling. The individual or user then places a second endpoint anchor on the opposite emotion where it begins and where it turns neutral. There are no intermediate numbers or delineation, it is based on spatially identifying the degree of separation. The individual may repeat this over a period of time, e.g. two weeks, or more to build an average of the emotion range of a neutral state and of an emotion state. The assessment thereof indicates the emotion ranges a particular individual may feel, thus personalizing an emotion state and adding flexibility into the understanding of an individual's emotion state.

In other aspects, the computer implemented system may further track the first and second endpoint anchor placement, along with speed of selection, whether or not the individual moved the anchor, including the degree of movement and the amount of movement, including over a time series set of assessments. Further, in other aspects, a third anchor point may be utilized to dictate how an individual felt, this anchor point may be placed in between the first and second endpoints to provide a more accurate assessment. Similarly, a fourth endpoint anchor may be added along with a first, second, and third to further constrain the range of emotion, for example to modify the neutral state range.

In the disclosed embodiments, the assessment and methods may be performed on private events such as emotions, pain, hunger, and health symptoms. The principles remain the same, wherein an emotion state dyad, often of opposite emotion states is formed, and endpoint anchors establish a zone of the emotion state and that of neutral. The assessment may be refined over repetitive trials in time series, with observations made about the individual's selection, and how said selection may evolve over time and over treatment or otherwise.

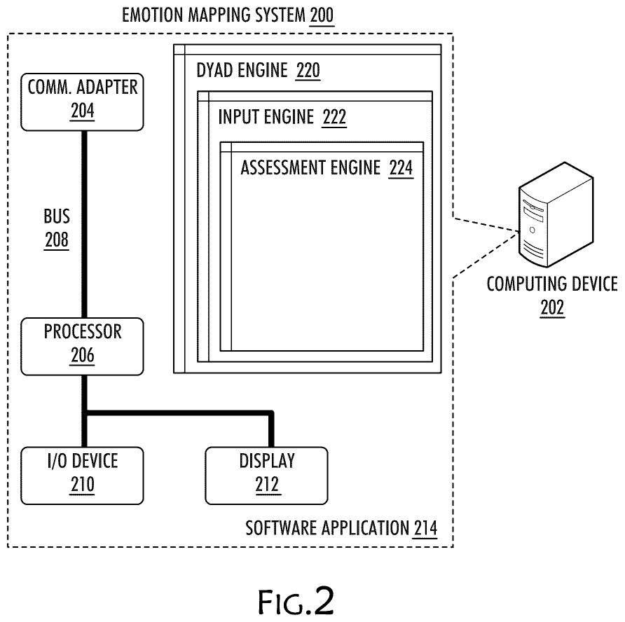

Referring now to FIG. 2, an example of an emotion mapping system. In the example a computing device 200 is configured with a software application 214, wherein the software application 214 is executed by a processor 206 that stores the contents of executable code within memory. In doing so the dyad engine 220 is stored on the memory of the computing device and is translated and executed at the processor 206, wherein the instructions travel along the bus from the memory and back to the processor. When a user interacts with the computing system 202 and application, through an I/O device or interface, the user transmits signals to the processor in respect to prompts or other features. In the case of the dyad engine, the word pair would be displayed with some degree of separation. The dyad engine may automatically populate emotion state combinations that most closely align to the individuals stated goals. These stated goals may be inferred from past experience with the system or may be entered by a professional such as a therapist or a computer scientist working to develop the testing assessment.

Continuing, the input engine receives input, often a first, second, and third anchor point (in other embodiments a fourth, fifth, sixth anchor endpoint may be established), wherein the anchor points may be selected by a mouse moving an anchor point to a specific location, thus defining the beginning of an emotion state, a neutral state, and, the opposite emotion state. This selection may be made by any number of input devices, including a touchscreen, a keyboard, through augmented reality, voice (including the use of natural language processing). As referenced earlier, when determining the hue of green, there is bodily sensation and categorization of such, and the emotion state may be further characterized by the individual. The input engine utilizes aspects of the individuals input, such as selection speed, change in selection, amount of change, time spent in selecting, or other aspects associated with selecting an endpoint. In combination, the dyad engine 220, and the selection engine 222, provide input for the assessment engine 224 to provide a neutral zone or state and an emotion zone or state that provides an assessment of an individual's emotion state, including a mapping.

Continuing, in one aspect the emotion region is detected by an individual selecting a first endpoint anchor while moving towards a neutral bodily sensation. The individual then selects a second endpoint anchor while moving aware from a second emotion, utilizing a bodily sensation. The two endpoint anchors are defined in the space between as a neutral region, wherein an individual's emotion is neutral. Lastly, the individual selects an emotion state and places an emotion anchor. This emotion anchor may appear in any degree of separation between the dyad pairs, and may be sampled with any timeline, including within minutes or days, or weeks, and then the assessment is built from the sampling data. This data is typically averaged or otherwise assimilated across multiple time series samples of endpoint anchors and emotion anchors. In doing so one may see a change in neutrality, an increase in various emotion regions, and where an individual places themselves in relation to the dyad pair.

Most assessments are highly structure, and that structure provides validity for standardization. However, the rigid structure fails to encompass the breadth of human emotion states, including the bodily sensation of an emotion state and respective categorization. The disclosure herein provides for a flexible presentation, that includes randomization and variation as a means to prove validity. For example, the following parameters may be randomized when conducting the mapping and presenting dyads: 1) Order of questions 2) Order of endpoint anchors 3) Amount of questions provided at one sitting 4) Length of space between dyads, or the degree of separation 5) Orientation of the separation between dyads (circle form, line form, etc.) 6) start point and response tick time. The variation of the above elements may be utilized to increase engagement, and retain validity of the representations made by individuals. Further benefits include reduction in memory effects, and flexibility of integrating the mapping assessment into other applications.

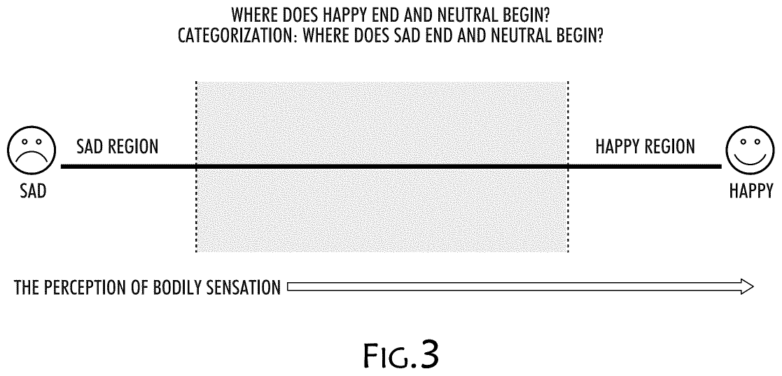

In FIG. 3, an illustration of an example of perception of bodily sensation with a dyad pair is disclosed. In the example an individual select's where happy begins and ends and where sad begins and ends. In the middle forms the neutral state of emotion. The perception of bodily sensation allows for categorization of the emotion state that is unique to each individual and that may give insight into an individual's perception. Examples of word dyads or dyad pairs include emotion states such as anxious/calm, sad/happy, fatigued/energized, insecure/confident, not control/control, fearful/brave, unsafe/safe, unsatisfied/satisfied, tense/relax, hated/loved, angry/compassionate, dirty/clean, hungry/full, sick/healthy, idle/busy, distract/focus, avoid/engage, chaos/predict, pessimistic/optimistic, cold/hot, pain/pleasure, weak/strong, to name a few.

In the example of FIG. 3, for the emotion dyad, the placement of an individual endpoint anchors determines the relative size of the emotion regions or zones or states, and the respective neutral zones. Further, the placement of an emotion anchor places an anchor that correlates with how the individual perceives bodily sensation at that given time. A score is formulated or otherwise an indicator or assessment value is generated based on time series responses and averaging of results, including the first and second endpoint anchors and the emotion anchor. In one aspect, a sample may occur with a dyad pair once a day, every day for at least two weeks, or may continue for months or years to validate results and continue monitoring an individual's emotion state. The result would be an average of scores and this would form a more accurate picture of where a person's emotion state zone occurs and where the neutral zone is.

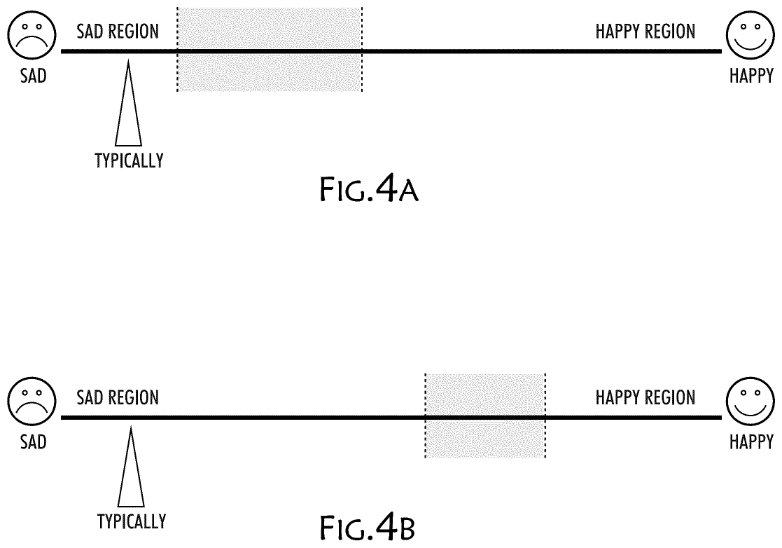

In the example depicted in FIGS. 4A and 4B, an example of a first and second endpoint and a neutral zone is indicated, along with an emotion anchor on where the individuals currently places their emotion state. In the example of FIG. 4A, the individual has a neutral zone closer to the sad emotion and has placed their emotion anchor closer to the neutral zone. In the example of FIG. 4B, the individual places a neutral zone closer to happy, and their respective response or emotion anchor further from happy.

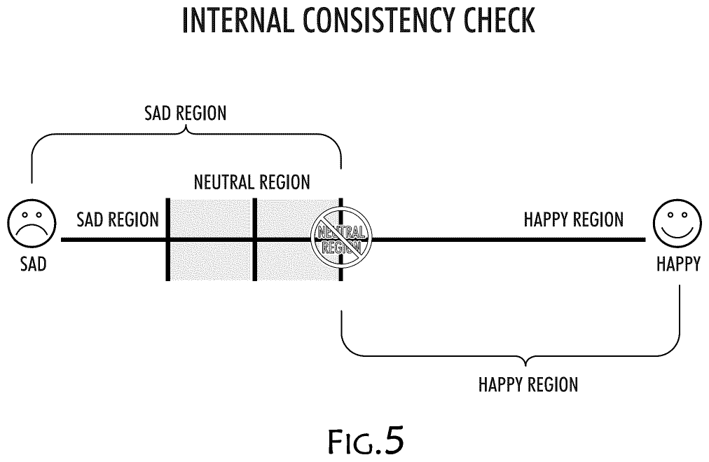

In the example of FIG. 5, an example of an internal consistency check is disclosed, wherein a sad region and a happy region are identified along with a neutral point, that is bounded by an individual's first endpoint and second endpoint. In this example, an individual is evaluated as to whether they are attending to the task. In doing so this shows an invalid neutral region, whereby the size of the neutral region would take up negative space. The relative size and placement of the neutral region informs us in real time whether the user is attending to the task. In similar aspects, a method of mapping the sad region and happy region may be applied to other emotion states and is based on at least the perceived bodily sensation, and may be validated based on the size of the respective neutral zone. In this regard, an individual being administered the computing system configured with a software application having a dyad engine, an input engine, and an assessment engine, may then take the user's input and place a score on the emotion that is more accurate and representative of an individuals perceived bodily sensation of the dyad pair. As will be appreciated, an internal consistency check can be utilized to validate the tool and to identify whether a user is attending to the task and ensure more accurate results. In some instances, the size of the neutral region can be measures and if its size (e.g. length, degree of separation, proportionality) is zero or below, an indication is provided that a user is improperly performing a given task. In some instances, an internal consistency check is done in real time.

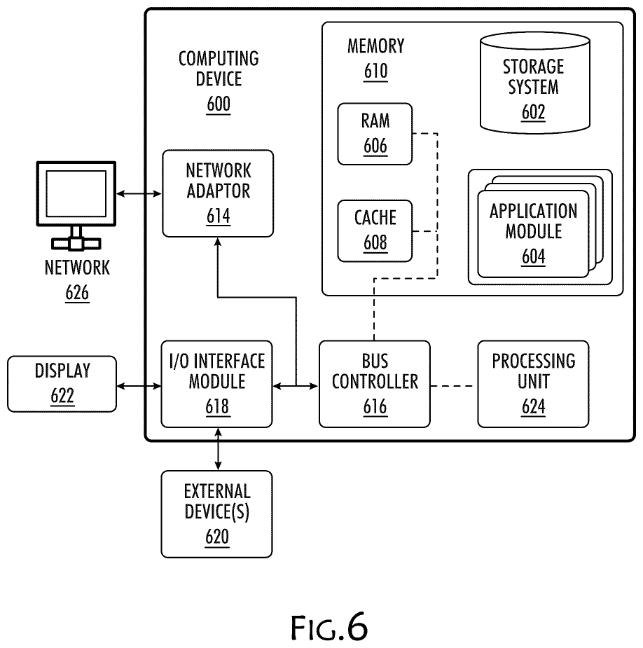

In the example of FIG. 6, a general-purpose computing device is disclosed. In other aspects a microcontroller may be adapted for specific elements of the disclosure herein or even further, a special purpose computing device may form elements of the disclosure. In the example embodiment of FIG. 6, the computing device is comprised of several components. In the example, the computing device is equipped with a timer. The timer may be used in applications such as applications for generating time delays for battery conservation or to control sampling rates, etc. The computing device is equipped with memory, wherein the memory contains a long-term storage system that is comprised of solid-state drive technology or may also be equipped with other hard drive technologies (including the various types of Parallel Advanced Technology Attachment, Serial ATA, Small Computer System Interface, and SSD). Further, the long-term storage may include both volatile and non-volatile memory components. For example, the processing unit and or engine of the application may access data tables or information in relational databases or in unstructured databases within the long-term storage, such as an SSD. The memory of the example embodiment of a computing device also contains random access memory (RAM) which holds the program instructions along with a cache for buffering the flow of instructions to the processing unit. The RAM is often comprised of volatile memory but may also comprises nonvolatile memory. RAM is data space that is used temporarily for storing constant and variable values that are used by the computing device during normal program execution by the processing unit. Similar to data RAM, special function registers may also exist, special function registers operate similar to RAM registers allowing for both read and write. Where special function registers differ is that they may be dedicated to control on-chip hardware, outside of the processing unit.

Further disclosed in the example embodiment of FIG. 6, is an application module. The application module is loaded into memory configured on the computing device. The disclosure herein may form an application module and thus may be configured with a computing device to process programmable instructions. In this example, the application module will load into memory, typically RAM, and further through the bus controller transmit instructions to the processing unit. The processing unit, in this example, is configured to a system bus that provides a pathway for digital signals to rapidly move data into the system and to the processing unit. A typical system bus maintains control over three internal buses or pathways, namely a data bus, an address bus, and a control bus. The I/O interface module can be any number of generic I/O, including programmed I/O, direct memory access, and channel I/O. Further, within programmed I/O it may be either port-mapped I/O or memory mapped I/O or any other protocol that can efficiently handle incoming information or signals.

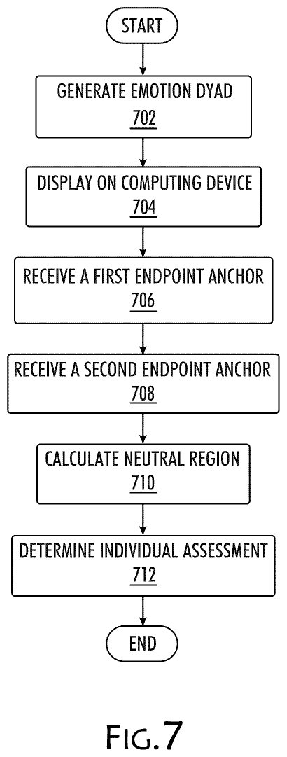

Referring now to FIG. 7, an example flow chat of a method for conducting mapping of an emotion state is disclosed. In the example a user or individual starts with the assessment by the computing device generating an emotion dyad, typically with a dyad engine and a list of potential dyad's relative to the individual or curated based on experience to the system (such as previous testing, or medical or clinical signs).

Once generated, the computing device displays the dyad word pairings, and displays a degree of separation between the word pairs that allows a user to place endpoints or anchors. Next, the individual places a first endpoint anchor leading from the emotion state to the feeling of neutrality. This order of operation may move from either word. Next, a second endpoint anchor is placed, from the feeling of the opposite second emotion state to a place of neutrality. The bounding of the first endpoint anchor and the second endpoint anchor delineate the neutral zone. Outside of the neutral zone is the emotion zone for each dyad. The computing system calculates the neutral zone and the emotion zones and stores the information within a data repository, often a relational database. The assessment may be repeated and the respective zones analyzed. In one aspect, an average may be taken of the first endpoint anchor and the average may be placed on a summary dyad pair that accounts for all trials. This may occur for the second endpoint anchor and the selected emotion state. Thus, the assessment engine may summarize the input received and provide an overall theme or time series response to an individual's emotion state, including how the emotion state is changing in response to treatment.

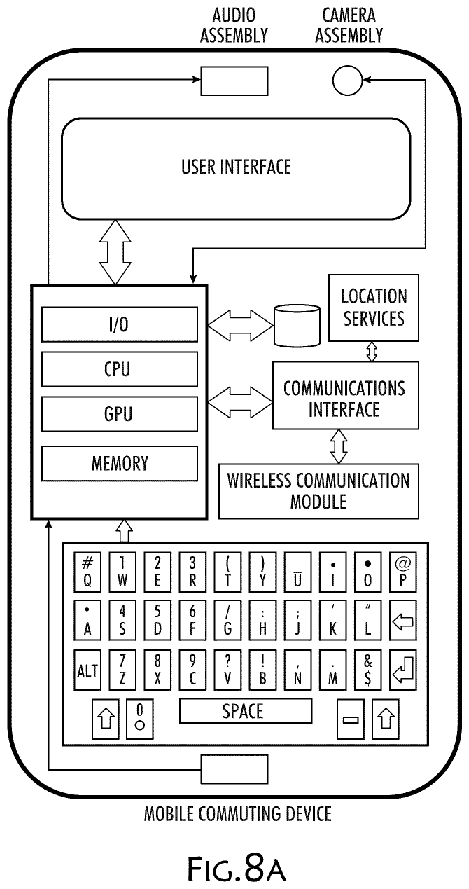

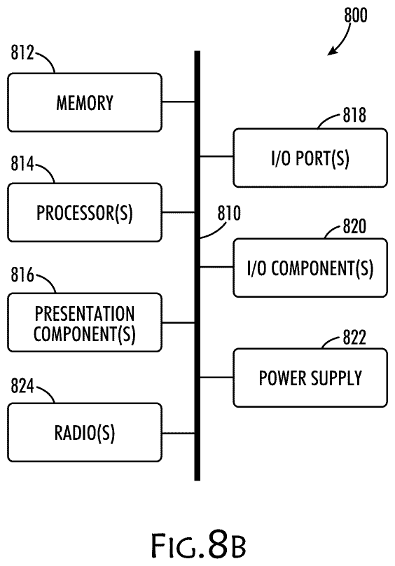

Referring now to FIG. 8A, an example of a computing device is disclosed. In the example an individual may experience or respond to the assessment on a mobile computing device, wherein a user's responses may be acquired outside of a clinical setting and this increase the potential for accurate feedback and placement. The computing device may include input via a touch screen interface and allow for placement via sliders or via anchors or other UX experiences that allow for easily responding to the dyad engine's prompt of word pairs.

FIG. 8B illustrates a block diagram of an example computing environment and/or device architecture in which some implementations of the present technology may be employed, and can be generally designated as computing device 800. Computing device 800 is merely one example of a suitable computing environment and is not intended to suggest any limitation as to the scope of use or functionality of the invention. Neither should the computing device 800 be interpreted as having any dependency or requirement relating to any one or combination of components illustrated.

Embodiments of the invention can be described in the general context of computer code or machine-useable instructions, including computer-executable instructions such as program modules, being executed by a computer or other machine (virtual or otherwise), such as a smartphone or other handheld device. Generally, program modules, or engines, including routines, programs, objects, components, data structures etc., refer to code that perform particular tasks or implement particular abstract data types. Embodiments of the invention can be practiced in a variety of system configurations, including hand-held devices, consumer electronics, general-purpose computers, more specialized computing devices, etc. Embodiments of the invention can also be practiced in distributed computing environments where tasks are performed by remote processing devices that are linked through a communications network.

Computing device 800 includes a bus 810 that directly or indirectly couples the following devices: memory 812, one or more processors 814, one or more presentation components 816, input/output ports 818, input/output components 820, and an illustrative power supply 822. In some embodiments, devices described herein utilize wired and rechargeable batteries and power supplies. Bus 810 represents what can be one or more busses (such as an address bus, data bus or combination thereof). Although the various blocks of FIG. 8B are shown with clearly delineated lines for the sake of clarity, in reality, such delineations are not so clear and these lines can overlap. For example, one can consider a presentation component such as a display device to be an I/O component as well. Also, processors generally have memory in the form of cache. It is recognized that such is the nature of the art, and reiterate that the diagram of FIG. 8 is merely illustrative of an example computing device that can be used in connection with one or more embodiments of the present disclosure. Distinction is not made between such categories as “workstation,” “server,” “laptop,” “hand-held device,” etc., as all are contemplated within the scope of FIG. 5 and reference to “computing device” or “user device.”

Computing device 800 typically includes a variety of computer-readable media. Computer-readable media can be any available media that can be accessed by computing device 800, and includes both volatile and non-volatile media, removable and non-removable media. By way of example, and not limitation, computer-readable media can comprise computer storage media and communication media.

Computer storage media include volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 800. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner at to encode information in the signal. By way of example, and not limitation, communication media includes wired media such as a wired network or direct-wired connection, and wireless media such as acoustic, RF, infrared, NFC, Bluetooth, cellular, and other wireless media. Combinations of any of the above should also be included within the scope of computer-readable media.

Memory 812 includes computer storage media in the form of volatile and/or non-volatile memory. As depicted, memory 812 includes instructions 824, when executed by processor(s) 814 are configured to cause the computing device to perform any of the operations described herein, in reference to the above discussed figures, or to implement any program modules described herein. The memory can be removable, non-removable, or a combination thereof. Illustrative hardware devices include solid-state memory, hard drives, optical-disc drives, etc. Computing device 800 includes one or more processors that read data from various entities such as memory 812 or I/O components 820. Presentation component(s) 816 present data indications to a user or other device. Illustrative presentation components include a display device, speaker, printing component, vibrating component, etc.

I/O ports 818 allow computing device 800 to be logically coupled to other devices including I/O components 820, some of which can be built in. Illustrative components include a microphone, joystick, touch screen, presentation component, satellite dish, scanner, printer, wireless device, battery, etc

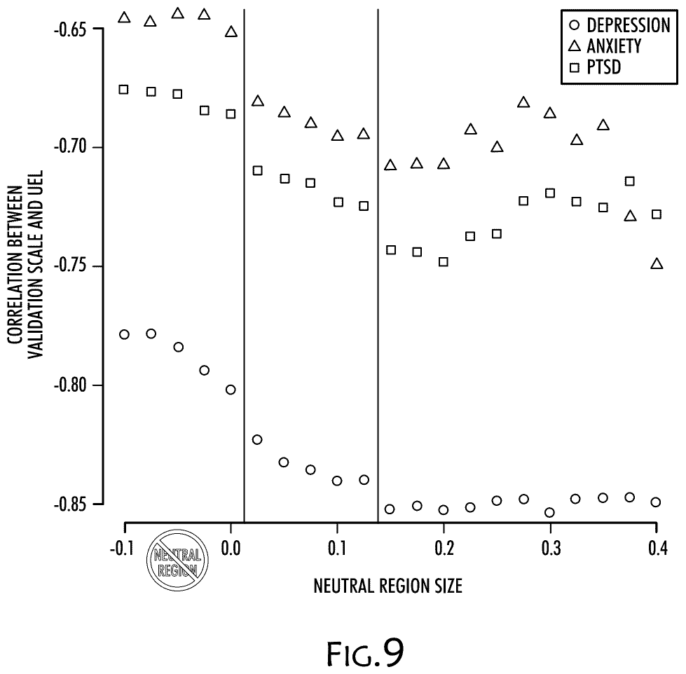

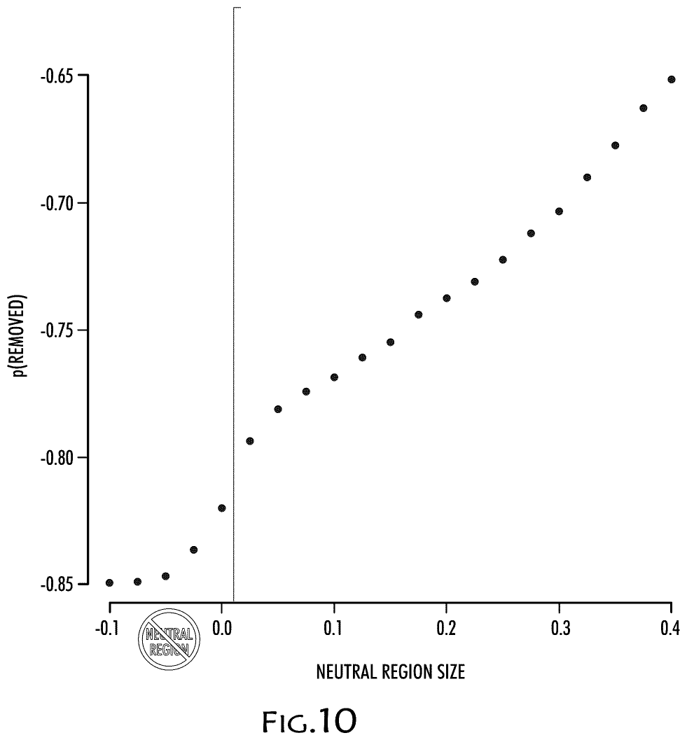

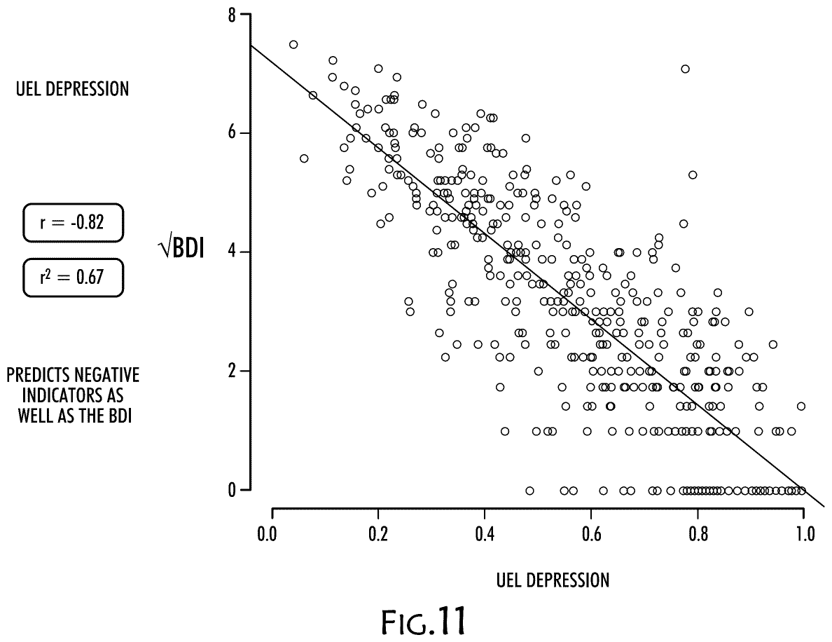

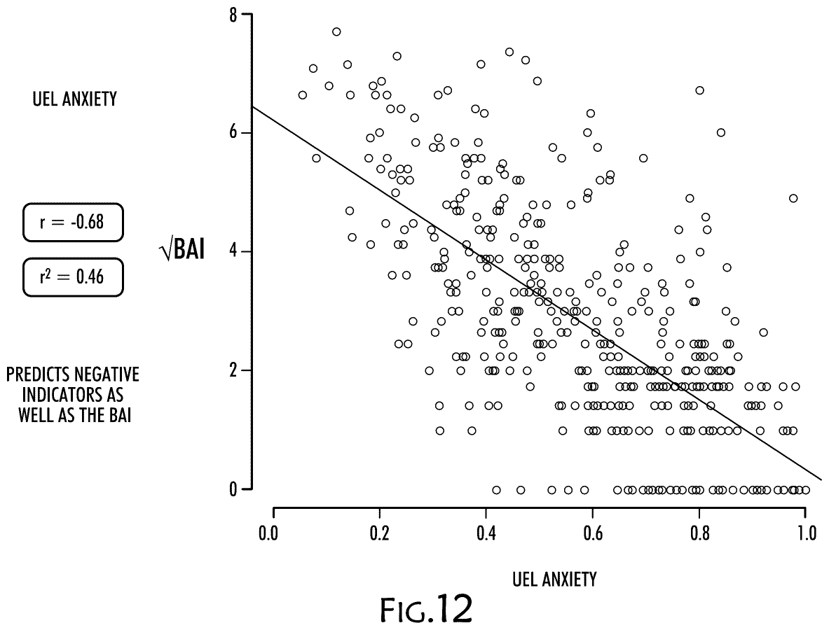

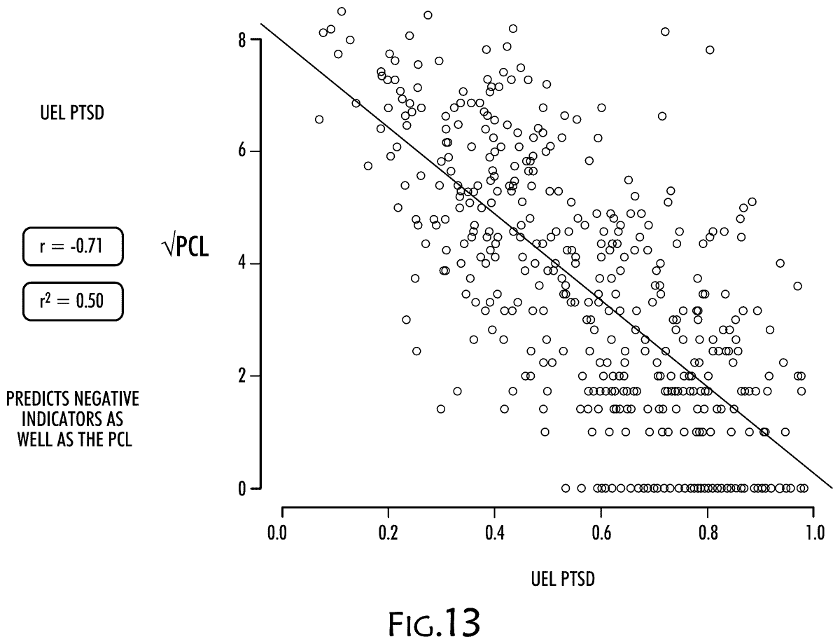

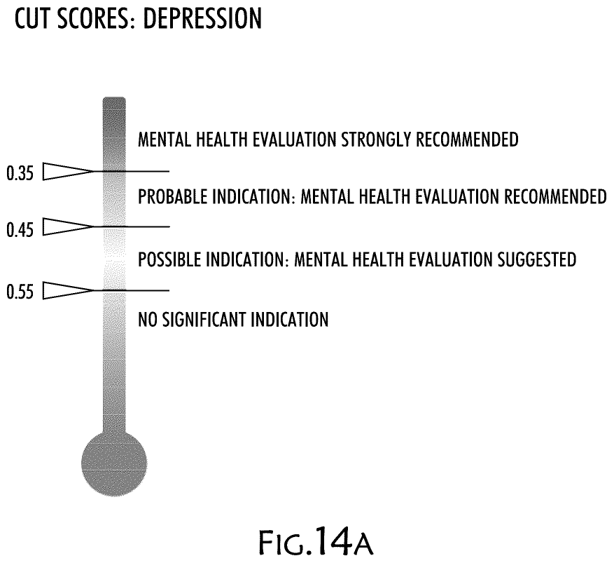

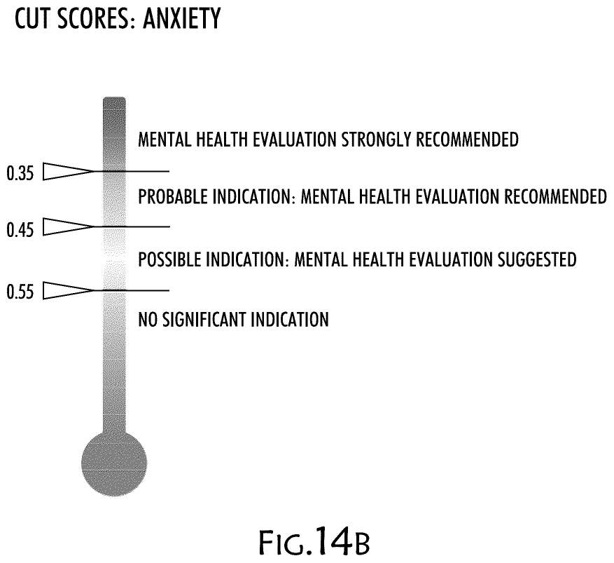

FIGS. 9-13 are illustrative of responses and trials conducted utilizing the systems and methods disclosed herein, and validation thereof. Such tests allow placement of an individual's typical feeling over a time period, the sizes of the emotion regions, and the sizes of the neutral regions. Further, the systems and methods herein allow capture of real time data, and may be applied at varying times or during varying periods of stress or treatment to understand an individual's emotion state. Example assessments include the ability to score for depression, anxiety, and post-traumatic stress disorder (PTSD). Further, in testing, these scores are validated and proved across the Beck Depression Inventory (BDI), Beck Anxiety Inventory (BAI), and PCL. For instance, referring to FIG. 9, illustrated is neutral region size and the correlation of the visual scale with validation measures, with lower being better. As depicted there are discontinuities at 0 and at around 0.13, and 0 being the chosen threshold for an inattentive user and/or measurement issue. Referring to FIG. 10, neutral region size is shown with respect to number of participants removed, and as will be appreciated a threshold of 0 does not remove a significant number of participants. FIG. 11 illustrates a validation test of the universal emotion line UEL Depression measure with the BDI (a widely used depression measure). As shown, the UEL is highly correlated with the BDI (−0.87) and that indicates a highly valid measure. FIG. 12 illustrates a validation test of the UEL Anxiety measure with the BAI (a widely used Anxiety measure). As shown, the UEL is highly correlated with the BAI (−0.68) and that indicates a highly valid measure. FIG. 13 illustrates a validation test of the UEL PtSD measure with the PCL (a widely used Anxiety measure). As shown, the UEL is highly correlated with the BAI (−0.68) and that indicates a highly valid measure.

Embodiments of the technology are further illustrated by way of the following example aspects. According to some aspects of the technology described herein systems and methods are provided for assessing emotion categorization and labeling utilizing a bounded visual analog scale (also referred to herein as a universal emotion line, emotion scale, visual scale). As will be appreciated, a visual analog scale can utilize received input from a user and further utilizes signals or information represented by a continuously variable spatial position or distance, for instance a spatial position and/or distance between two endpoint anchors or bounding points. The bounded visual scale or universal emotion line additionally can be implemented to measure how people categorize emotions and how that categorization related to their reported magnitude of underlying emotional affect.

The universal emotion line generates and presents a bounded line segment that is labeled on the left and right boundary with an emotion dyad of opposing emotions (i.e., happy-sad). Twelve emotion dyads were presented in the universal emotion line: angry-compassionate; anxious-calm; cold-hot; fatigued-energized; fearful-brave; not in control-in control; powerless-powerful; sad-happy; tired-awake; uninterested-excited; unsafe-safe; and unsatisfied-satisfied.

Questions may be presented (e.g. five questions) for every emotion dyad. Two questions assessed how the participant categorized emotions. These two boundary questions asked participants to indicate where one emotion of the dyad ends and neutral begins (i.e., Please move the line of the spectrum to the point where happy feelings end and neutral feelings begin”). Three questions assessed the participant's emotional experiences. These three time questions asked participants to indicate how they felt during three temporal reference points: now, typically, and the past two weeks (i.e., Please move the line on the spectrum to the point that indicates how you typically feel”). The three temporal references can be selected to measure both current emotional states (i.e., now), as well as more generalized mood states (i.e., typically, two weeks, etc.).

In some example embodiments a universal emotion line can be implemented to determine underlying changes in emotion categorization and labeling. In some instances, additional variables may be generated and analyzed, for example neutral region and typical affective state. The neutral region represents the size of the neutral area between the positive and negative emotion dyad (i.e. bounded by endpoints or anchors), calculated by subtracting the negative boundary from the positive boundary. The typical affective state represents a user's emotional state at a given time or averaged across how they typically feel and how they felt over a period of time (e.g. the last two weeks).

In some further example embodiments, changes in a typical affective state can be related to changes in emotion classification for a given dyad. An additional variable, an emotion label, can be utilized to categorize a user's typical affective state based on the placement of their boundaries for each emotion dyad. Specifically, a user's typical affective state may be labeled as positive if it was placed above their positive emotion boundary, labeled as negative if it was placed below the negative emotion boundary, and labeled neutral if it was placed in between the emotion boundaries in the neutral region.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the scope of the invention. Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A computer implemented method for emotion mapping and assessment thereof, comprising:
(a) generating, by a computing device, an emotional dyad, wherein the emotional dyad comprises two different emotion states;
(b) displaying, by the computing device, the emotional dyad, wherein displaying places the emotional dyad with a degree of separation;
(c) receiving, by the computing device from an individual, a first endpoint anchor on the emotional dyad, the first endpoint anchor indicating when the individual moves from a first emotion state to a first neutral state;
(d) receiving, by the computing device from the individual, a second endpoint anchor on the emotional dyad, the second endpoint anchor indicating when the individual moves from a second emotion state to a second neutral state;
(e) receiving, by the computing device from the individual, an emotion anchor within the degree of separation; and
(f) calculating, by the computing device, the length of separation from the first endpoint anchor and the second endpoint anchor, wherein the length of separation is a neutral region, and lengths outside of the neutral region are emotion regions, as well as a location of the emotion anchor.

2. The method of claim 1, further comprising determining the individual's assessment by averaging the neutral region and the emotion anchor in one or more cycles of (a)-(f).

3. The method of claim 1, wherein generating an emotional dyad generates a first emotion state separated by a length from a second emotion state.

4. The method of claim 1, further comprising steps (a)-(f) at least one time per day over at least a 14-day period.

5. The method of claim 1, wherein determining the individual assessment further comprises calculating an average of the emotion regions, an average of the first and second endpoint anchors, and an average of the emotion anchor.

6. A system for emotion mapping and assessment thereof, comprising:
a computing device, equipped with a user input device and a display device;
a software application held on memory of the computing device, the software application comprising:
(a) a dyad engine, wherein the dyad engine generates dyad pairs;
(b) an input engine, wherein the input engine accepts user input of a first anchor endpoint and a second anchor endpoint, and an emotion anchor; and
(c) an assessment engine, wherein the assessment engine calculates an emotion state based on the dyad engine and the input engine.

7. The system of claim 6, wherein the input device is a computing mouse, or a touchscreen, or a keyboard.

8. The system of claim 6, wherein the dyad engine generates dyad pairs that are opposite emotion states.

9. The system of claim 6, wherein the input engine receives a third anchor endpoint and a fourth anchor endpoint.

10. The system of claim 6, wherein the input engine receives a second emotion anchor and a third emotion anchor.

11. The system of claim 6, wherein the assessment engine utilizes a neutral zone and an emotion zone along the dyad pairs, along with an average of the emotion anchor to determine the individual's emotion state.
